(19) United States
(12) Patent Application Publication
(10) Pub. No.: US 2012/0116479 A1
(43) Pub. Date: May 10, 2012

(54) TWO-WIRE MEDICAL IMPLANT CONNECTION

(76) Inventors: Werner Meskins, Opwijk (BE); Tony Nygard, Terrigal (AU)

(21) Appl. No.: 12/941,744

(22) Filed: Nov. 8, 2010

(51) Int. Cl. A61N 1/36; A61F 11/04

(52) U.S. Cl. 607/57; 607/60

(57) ABSTRACT

A two-wire medical implant, method and system for transferring power and data over a two-wire connection between a first medical implant and a second medical implant. The second medical implant comprises a clamping circuit for extracting the data. In one form, the second medical implant also comprises a voltage multiplier which is formed in part by the clamping circuit. In one embodiment, the second medical implant also comprises a DC decoupling capacitor which forms a part of the clamping circuit. The medical implant and medical implant system may be used in a cochlear implant system.

### BACKGROUND

1. Field of the Invention

The present application relates generally to a medical implant, and more particularly, to a two-wire connection for a medical implant and a method for transferring power and data between two or more medical implants.

2. Related Art

Medical implants require power to operate and perform intended functions. Sometimes this power may be provided from an external source, but in some cases, the power is provided by an internal power source such as a battery.

In some devices, it is necessary to transfer this power to different parts of the device, or to different modules of the device. Energy storage and power transfers used to drive operational circuits are usually in direct current (DC) form. When transfer or transmission of electrical power is performed within the body of a recipient of the medical implant, it is important to avoid or at least minimise any contact with tissue, since DC current flowing through tissue can have deleterious effects to the tissue as will be understood by the person skilled in the art.

In some applications, the transfer of data is also performed over the same link. To reduce the risk of DC components coming into contact with the user's tissue, special coding may be used to ensure that the data signal being transmitted is “DC free”.

One particular medical device in which such power transfer may be used is a cochlear implant. A cochlear implant allows for electrical stimulating signals to be applied directly to the auditory nerve fibres of the patient, allowing the brain to perceive a hearing sensation approximating the natural hearing sensation. These stimulating signals are applied by an array of electrodes implanted into the patient's cochlea.

The electrode array is connected to a stimulator unit (by way of a lead) which generates the electrical signals for delivery to the electrode array. The stimulator unit in turn is operationally connected to a signal processing unit which also contains a microphone for receiving audio signals from the environment, and for processing these signals to generate control signals for the stimulator. In many cases, the stimulator unit is, in use, implanted into the recipient, while the signal processing unit is located external to the recipient. The functions performed by the stimulator unit implanted within the recipient require power.

In some cases, a medical implant system will comprise two or more implanted devices, which may be active implantable medical devices (AIMDs). One of these may contain a power source and data generator, which are to be transferred by wire to one or more other AIMDs.

### SUMMARY

In one aspect, a medical implant for connection to a two-wire connection is provided. In one form, the medical implant comprises a clamping circuit for extracting data from a signal received by the medical implant from the two-wire connection. The clamping circuit also provides a rectifying function.

In another aspect, a two-wire medical implant system is provided. The system comprises a first medical implant and a second medical implant. The first medical implant comprises a power source and a data source. A two-wire connection connects the first implant to the second implant and carries a signal between the two implants. The second implant comprises in one form, a clamping circuit for extracting data from the signal received by the medical implant from the two-wire connection. The clamping circuit also provides a rectifying function. The clamping circuit may also be DC decoupled by a DC decoupler.

In one form, the second medical implant also comprises a power storage device such as a capacitor, for storing power from the rectified signal.

In another aspect, a cochlear implant system is provided, which comprises an external component and an internal component. The internal component comprises a first medical implant and a second medical implant connected via a two-wire connection. The second medical implant comprises a clamping circuit for extracting data from a signal received on the two-wire connection, as well as a stimulator for stimulating the user.

In a further aspect, a medical implant for connection to a two-wire connection is provided, which comprises a power storage device for storing power received by the medical implant, as well as a clamping circuit for extracting data on the signal on the two-wire connection.

In another aspect, a method of processing a signal on a two-wire connection of a medical implant or medical implant system is provided. The signal may have a power component and a data component for transfer to one or more medical implants in the medical implant system. The method involves receiving the signal, rectifying the signal using a rectifier to extract the power component and clamping the signal using the rectifier to extract the data component.

### BRIEF DESCRIPTION OF THE DRAWINGS

FIG. 1 shows an exemplary medical implant system to which various aspects of the present disclosure may be applied;

FIG. 2 shows a cochlear implant system to which various aspects of the present disclosure may be applied;

FIG. 3 shows a representation of a medical implant system with DC decoupling capacitors, in accordance with an embodiment of the present invention;

FIG. 4A shows a general arrangement for a medical implant, in accordance with an embodiment of the present invention;

FIG. 4B shows a specific example of the arrangement of FIG. 4A, in accordance with an embodiment of the present invention;

FIG. 5A shows another general arrangement for a medical implant, in accordance with an embodiment of the present invention;

FIG. 5B shows a specific example of the arrangement of FIG. 5A, in accordance with an embodiment of the present invention;

FIG. 6A shows a medical implant with one example of a clamping circuit, in accordance with an embodiment of the present invention;

FIG. 6B shows a medical implant with another example of a clamping circuit using two capacitors, in accordance with an embodiment of the present invention;

FIG. 6C shows a medical implant with another example of a clamping circuit using a transformer, a diode and a capacitor, in accordance with an embodiment of the present invention;

FIG. 7 shows a medical implant in which the DC coupling capacitor forms part of the clamping circuit, in accordance with an embodiment of the present invention;

FIG. 8A shows a medical implant with one example of a clamping circuit in which a DC decoupling capacitor forms part of the clamping circuit, in accordance with an embodiment of the present invention;

FIG. 8B shows a medical implant with another example of a clamping circuit in which two DC decoupling capacitors form part of the clamping circuit, in accordance with an embodiment of the present invention;

FIG. 8C shows a medical implant with another example of a clamping circuit using a transformer, a diode and a DC decoupling capacitor, in accordance with an embodiment of the present invention;

FIG. 9A shows a general arrangement for a medical implant with a voltage multiplier circuit using a DC decoupling capacitor, in accordance with an embodiment of the present invention;

FIG. 9B shows a specific example of the arrangement of FIG. 9A, in accordance with an embodiment of the present invention;

FIG. 10 shows a medical implant with an example voltage doubler circuit for use in the arrangement of FIG. 8A, in accordance with an embodiment of the present invention;

FIG. 11 shows a medical implant with an example voltage doubler circuit for use in the arrangement of FIG. 8B, in accordance with an embodiment of the present invention;

FIG. 12 shows a medical implant with an example voltage doubler circuit for use in the arrangement of FIG. 8C, in accordance with an embodiment of the present invention;

FIG. 13 shows a medical implant with an example of a DC voltage rectification circuit such as a full-wave bridge rectifier, in accordance with an embodiment of the present invention;

FIG. 14A shows a medical implant with an example of a voltage quadrupler circuit, in accordance with an embodiment of the present invention;

FIG. 14B shows a medical implant with an example of another voltage quadrupler circuit, in accordance with an embodiment of the present invention;

FIG. 15 shows an example of a medical implant system using the voltage multiplier circuit of FIG. 11, in accordance with an embodiment of the present invention;

FIG. 16 shows the forward link in power and data transfer in the arrangement of FIG. 15, in accordance with an embodiment of the present invention;

FIG. 17 shows a medical implant system capable of back link operation, in accordance with an embodiment of the present invention;

FIG. 18 shows the back link data transfer in the arrangement of FIG. 17, in accordance with an embodiment of the present invention;

FIG. 19 shows a more detailed circuit arrangement for the arrangement shown in FIG. 17 without back link functionality, in accordance with an embodiment of the present invention;

FIG. 20 shows an example UART data frame structure for the signal to be carried by the two-wire connection, in accordance with an embodiment of the present invention;

FIG. 21A shows the received and reconstructed signal in the second medical implant in the arrangement of FIG. 19, in accordance with an embodiment of the present invention;

FIG. 21B shows the same received and reconstructed signal in the second medical implant shown in FIG. 21A but seen on a larger timescale, in accordance with an embodiment of the present invention;

FIG. 22 shows a representation of a forward link and backward data link between two implants, in accordance with an embodiment of the present invention;

FIG. 23 shows the interleaved transfer of forward and backward packets over a two-wire link between two implants, in accordance with an embodiment of the present invention;

FIG. 24 shows an arrangement for the medical implant system using a transformer, in accordance with an embodiment of the present invention;

FIG. 25 shows an example of a Manchester IEEE 802.3 encoded UART frame, in accordance with an embodiment of the present invention;

FIG. 26 shows the received waveform of the data signal on the second implant of a Manchester coded audio frame, in accordance with an embodiment of the present invention;

FIG. 27 shows the received waveform of the data signal at start-up on the second implant of a Manchester coded audio frame, in accordance with an embodiment of the present invention; and

FIG. 28 shows a cochlear implant system, in accordance with an embodiment of the present invention.

### DESCRIPTION

FIG. 1 shows an example of a medical implant system 500, comprising in this example, a first medical implant 100 and a second medical implant 200, connected to each other via a two-wire connection or lead 50. Medical implants 100, 200 could be any active implantable medical devices (AIMDs), such as for use in a cochlear implant system for example.

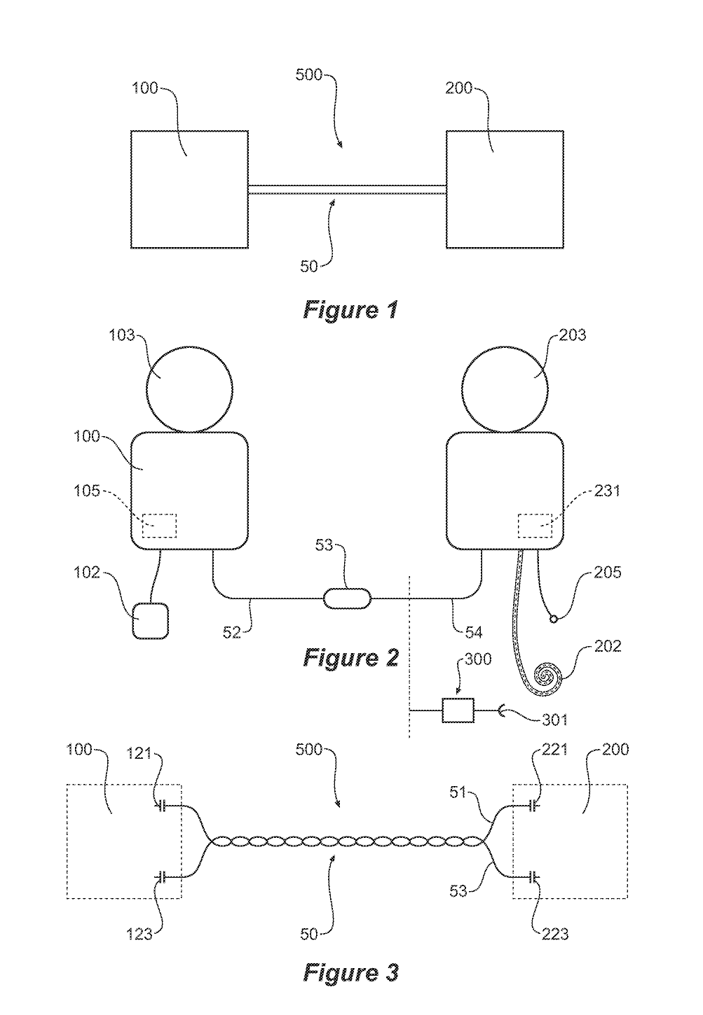

FIG. 2 shows one possible arrangement for a cochlear implant system 500 comprising in this example, first medical implant 100 which could be an implant containing a power source 105 such as a Li-ion battery, and may also support a microphone 102 for receiving audio signals from the surrounding environment. First medical implant 100 may also have a coil 103 for receiving charging power from an external source to keep power source 105 charged.

In some embodiments, first medical implant 100 may generate data in response to input from microphone 102. This data may be used to control the generation of stimulation signals generated by second medical implant 200, which in one example could be a cochlear nerve stimulator 200 using a stimulating electrode 202. Alternatively, special arrangements could be made to replace the second medical implant 200 by an actuator 300 such as a piezoelectric or electromechanical device anchored 301 with the auditory ossicles or in direct contact to the cochlea as a Direct Acoustic Cochlear Stimulation (DACS) system or skull as a Transcutaneous Bone Anchored Hearing Aid (TBAHA) system. FIG. 2 shows a representation of stimulator 200 being replaced by the DACS actuator 300 with anchor 301.

Two-wire lead 50 may include a connector 53 connecting first medical implant 100 with second medical implant 200 through which power and data may be transferred. Two-wire lead 52 will connect first medical implant 100 to connector 53 and two-wire lead 54 will connect connector 53 to second medical implant 200. In this case, the power from power source 105 may be transferred via connector 53 to charge a power storage device 231, which supplies power to the functional elements of the stimulator 200, including stimulating electrode 202.

In some embodiments, stimulator 200 may also have its own charge coil 203. Reference electrode 205 may also be provided.

In other embodiments, the data source in first medical implant 100 may be obtained from an external device such as a processor, rather than (or in conjunction with) microphone 102.

Many such medical implant systems will have one or more DC decouplers, such as DC decoupling capacitors 121, 123, 221 and 223 at the end of the two-wire connection lines when connected, as shown in FIG. 3. These DC decoupling capacitors keep the two-wire connection DC free, thus reducing the risk of tissue damage should insulation failure occur to the connector 53 or either of the two wires 51 or 53 of two-wire lead 50. The combined power and data signal delivered by the first medical implant 100 is placed over the DC decoupling capacitors 121, 123 and has a square or rectangular wave shape depending on the contents of the data to be transferred.

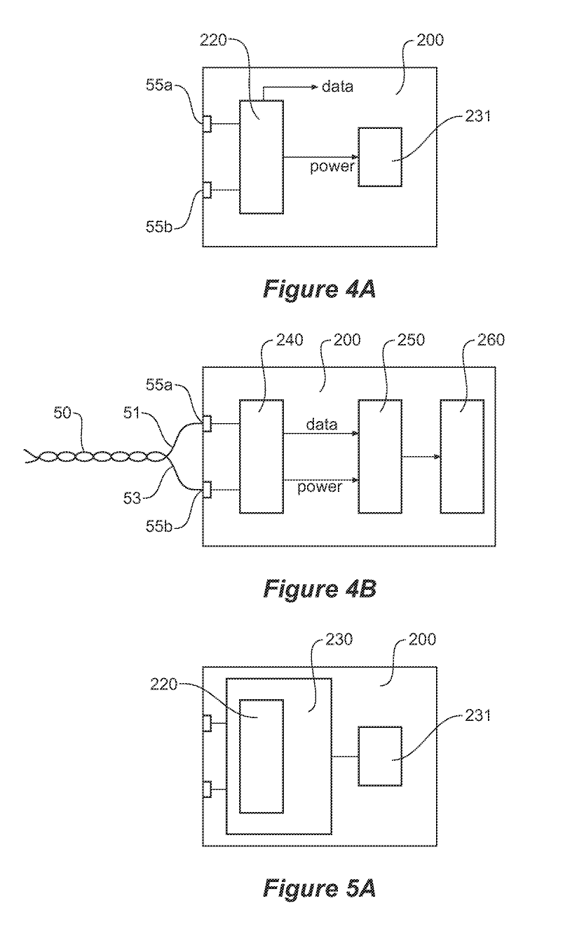

In one aspect, as shown in FIG. 4A, second medical implant or stimulator 200, comprises power storage device 231 for storing power received by the second medical implant via a signal on the two-wire lead (not shown in this view), and a clamping circuit 220 for extracting data received by the medical implant or stimulator 200 via the signal on the two-wire connection. Connector ports 55a and 55b are provided to allow connection of the medical implant 200 to the two-wire connection. In some embodiments, clamping circuit 220 also provides a rectification function for rectifying the signal on the two-wire lead.

FIG. 4B shows the second medical implant 200, comprising a two-wire power and data unit 240 which comprises the clamping circuit 220, extracting power and data from the two-wire 50 lead comprising wires 51 and 53 connected via ports 55a and 55b, a stimulator circuit 250 and a stimulator element or actuator 260.

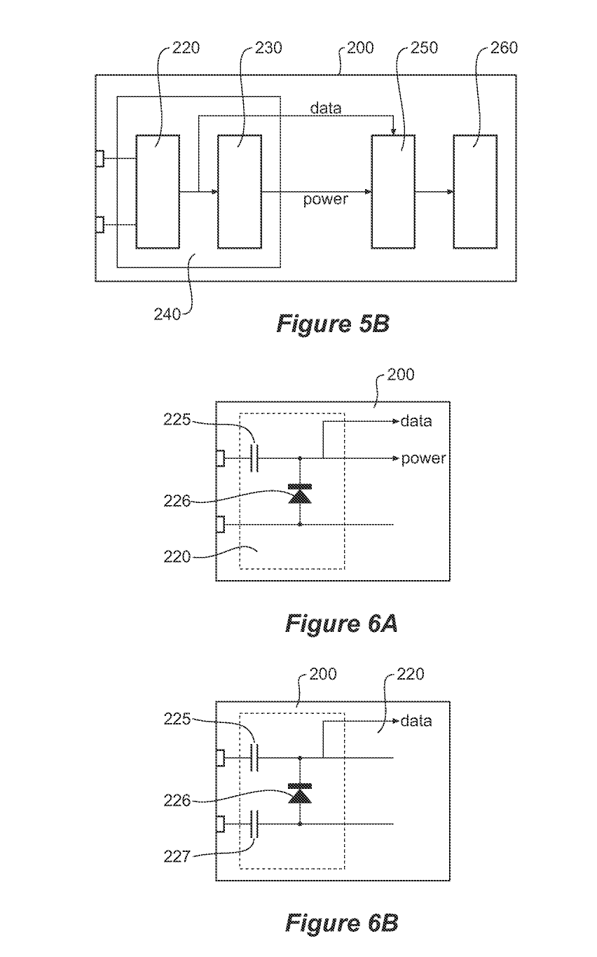

In another aspect, the clamping circuit 220 forms part of a voltage multiplier circuit 230 for multiplying the signal received on the two-wire lead 50 (not shown in this view), and for extracting and providing the power from the signal to power storage device 231 as shown in FIG. 5A. Again, in some embodiments, clamping circuit 220 also provides a rectifying function to rectify the signal on the two-wire connection.

A more detailed view of the two-wire power and data unit 240 is depicted in FIG. 5B. Shown there is the clamping circuit 220 for extracting data received by the medical implant or stimulator 200 via the signal on the two-wire lead 50 cascaded by the voltage multiplier circuit 230. The power and data unit 240 may further contain a power storage device for further voltage rectification and storing power (e.g. a tantalum capacitor) received by the medical implant via a signal on the two-wire lead and supplying power to the stimulator circuit 250.

The provision of the clamping circuit 220 provides for efficient data extraction from the signal, and in this arrangement, removes the need to provide DC-free or line coding in the first medical implant 100 generating the data. This aspect will be described in more detail below. A clamping circuit places either the positive or negative peak of a signal at a desired level, by adding or subtracting a DC component to or from the signal. Whether the DC component is added or subtracted may be determined by the polarity of a diode used in the clamping circuit.

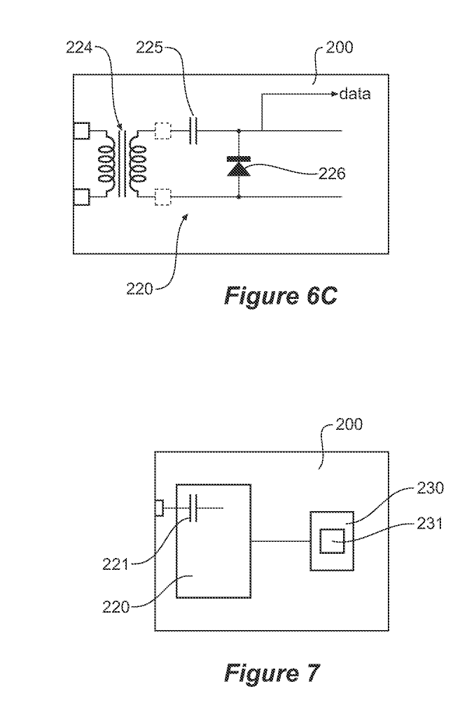

In one embodiment, as shown in FIG. 6A, the clamping circuit 220 is provided by a capacitor 225 connected to a first diode 226. In another embodiment as shown in FIG. 6B, clamping circuit 220 may be provided by a capacitor 225, a further capacitor 227, and first diode 226. In yet a further embodiment, clamping circuit 220 may be provided by capacitor 225, first diode 226 and transformer 224, as shown in FIG. 6C. Each of these embodiments provide for rectification of the signal on the two-wire connection.

In another aspect, the clamping circuit 220 is formed in part using a DC decoupler such as at least one DC decoupling capacitor as shown in FIG. 7. In this arrangement, it can be seen that medical implant 200 comprises clamping circuit 220 and a power circuit 230 containing a power storage device 231. In this arrangement, DC decoupling capacitor 221 (previously described with reference to FIG. 3), forms part of the clamping circuit 220. This provides for a more efficient use of components, saving on space and cost.

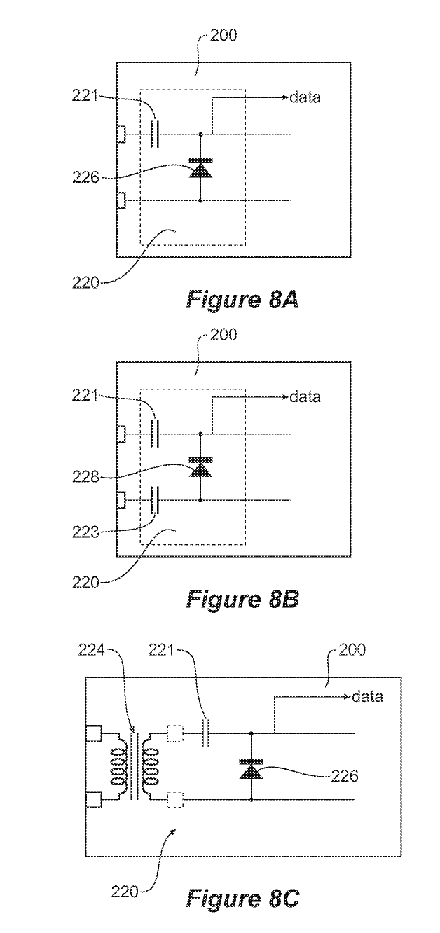

FIGS. 8A to 8C show the arrangements of FIGS. 6A to 6C with the capacitor 225 replaced with DC decoupling capacitor 221. In one particular example shown in FIG. 8B, both capacitors used to form clamping circuit 220 are provided by DC decoupling capacitors 221 and 223 to provide even greater efficiency of design. Of course, any other combination may be used, such as using DC decoupling capacitor 221, with a further capacitor 227 in place of DC decoupling capacitor 223. Again, these various embodiments of clamping circuit 220 also act as a rectifier.

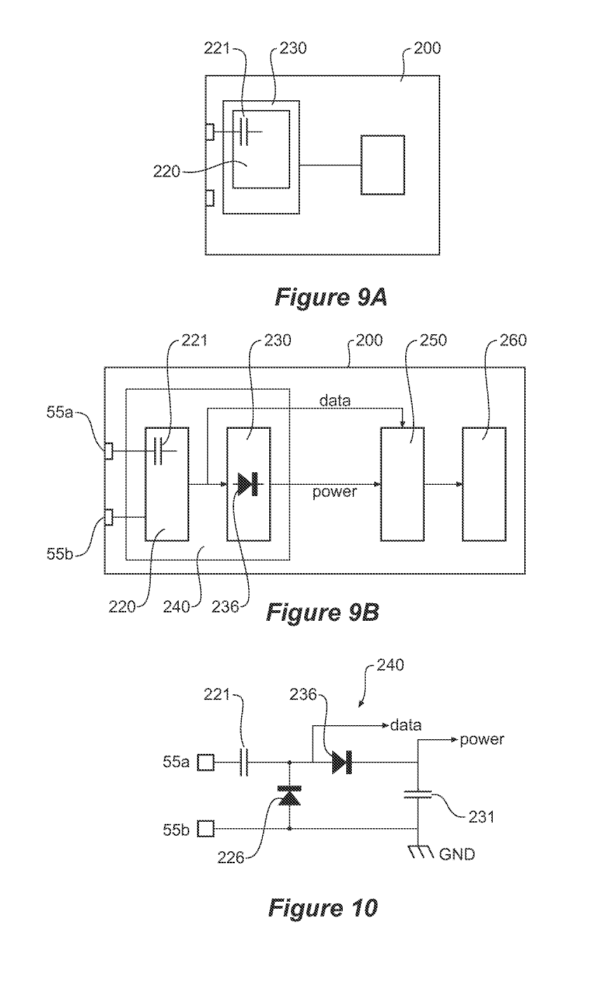

In another aspect, following from the arrangement of FIG. 5, the voltage multiplier circuit 230 may be formed in part by DC decoupling capacitor 221, which also forms part of clamping circuit 220, as shown in FIG. 9A.

In FIG. 9B, the two-wire power and data circuit 240 has a power circuit/voltage multiplier 230 containing a DC voltage rectification circuit that is connected to a DC decoupling capacitor 221, which also forms part of clamping circuit 220. The DC voltage rectification circuit supplies power to the stimulator circuit 250.

FIG. 10 shows a power and data unit 240 with voltage multiplier circuit 230 as a particular example of a rectification circuit, formed by DC decoupling capacitor 221, first diode 226 and second diode 236 and a power storage device 231 (e.g. a capacitor). In this figure, it can be seen where the received data extracted from the signal on the two-wire connection is accessed, as well as the power extracted from the signal on the two-wire connection, for storage in power storage device 231. The circuit shown in FIG. 10 is a voltage doubler, which will provide a DC signal with twice the peak magnitude of the input signal to provide power to the stimulator circuit 250 as will be appreciated by the person skilled the art.

FIG. 11 shows a medical implant 200 with two-wire connection, with the two DC decoupling capacitors 221, 223 and a first diode 226 forming the clamping circuit 220, and a second diode 236 and a power storage device 231 as parts of the power circuit 230.

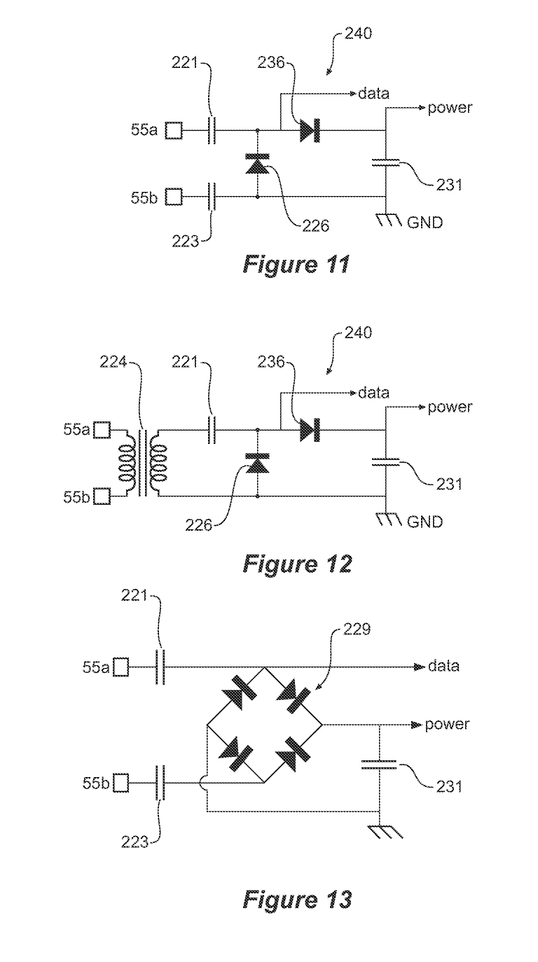

FIG. 12 shows another alternative in which a transformer 224 is used in place of DC decoupling capacitor 223, in conjunction with DC decoupling capacitor 221 and first diode 226 and second diode 236. The use of a transformer mitigates the risk of AC-leakage currents inside the human tissue likely to occur between the first and second implant whenever the stimulator element or actuator of the second implant are electrodes.

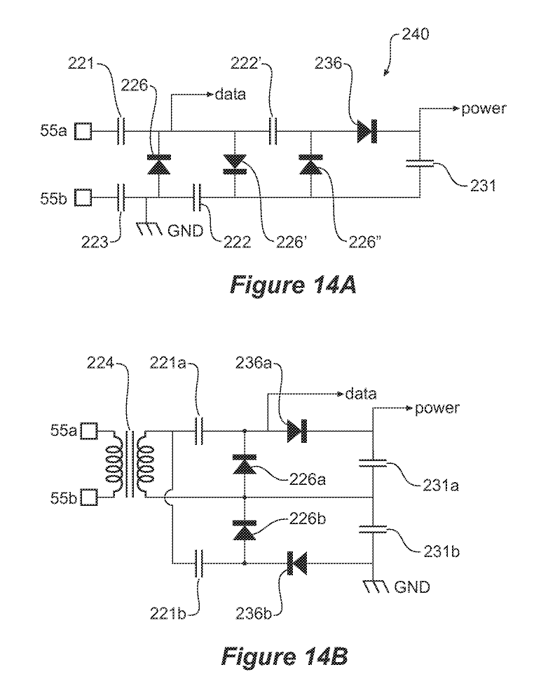

Other DC voltage rectification circuits such as a full-wave bridge rectifier 229 as shown in FIG. 13 or other multipliers may also be used, such as a voltage tripler or a voltage quadrupler, as shown in FIGS. 14A and 14B.

FIG. 13 shows input connectors 55a, 55b connected to DC decoupling capacitors 221 and 223 and full-wave bridge rectifier 229, providing power to storage capacitor 231.

FIG. 14A shows a voltage quadrupler made from diodes 226, 226′, 226″ and 236 and capacitors 221, 223, 222 and 222′. This provides the clamping function as previously described and a voltage quadrupling function to provide four times the input voltage to storage capacitor 231.

FIG. 14B shows another voltage quadrupling arrangement, with diodes 226a, 226b, 236a and 236b and capacitors 221a and 221b. In this arrangement, the voltage is stored over two storage capacitors 231a and 231b. This particular arrangement may be used with transformer 224, interfacing with the two wire connection via connector ports 55a and 55b.

Of course, one or more diodes may be replaced by MOSFET switches as done in synchronous rectification as will be appreciated by the person skilled in the art.

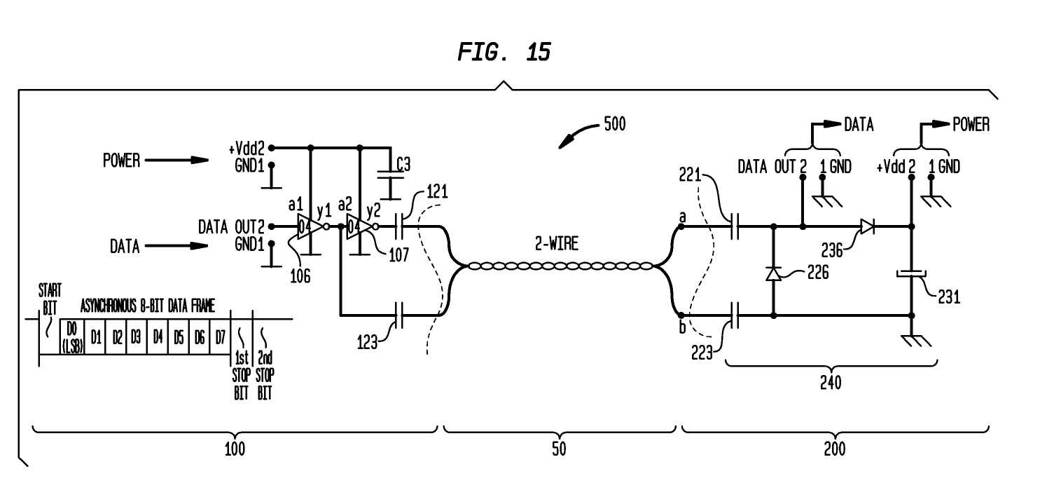

FIG. 15 shows a medical implant system 500 comprising first medical implant 100 and second medical implant 200, connected via two-wire lead 50. In this example, second medical implant 200 comprises power and data unit 240 including the voltage multiplier circuit formed in part by clamping circuit as well as second diode 236, which in turn is formed in part by DC decoupling capacitors 221 and 223. This is the arrangement described previously in relation to FIG. 11 for example. This arrangement also acts as a rectifier circuit.

It will be appreciated that a medical implant system 500 may also comprise further medical implants, connected in parallel to points a and b shown in FIG. 15. For example, the medical implant system 500 could comprise 2, 3, 4, 5, 6, 7, 8, 9, 10, 11, or more medical implants.

First medical implant 100 comprises a power source (not shown in this view), providing power between points Vdd 2 and gnd 1. This power source may be provided by a battery, such as a Li-ion battery, providing for example, about 3.6V, or the power source may be provided via a charge coil (not shown in this view) as previously described with reference to FIG. 2.

Also provided are two drivers 106, 107 (for example, provided by two logic inverter gates such as 74AC04 TTL logic or 74LVC04 low-voltage CMOS logic inverters, which provide a full bridge, or H-bridge. These drivers are supplied directly from the Li-ion battery for example, with a typical voltage between about 3.5 to about 4.1V.

The provision of the clamping circuit in the second medical implant 200, in the above arrangement allows for both power and data to be transferred over two wires, instead of the usual four wires as required in the prior art. Furthermore, the combined data and power signal over the 2-wire connection 50 does not have to be DC-free encoded (for example UART or I²C), thus no line coding is needed. It will be appreciated that DC-free encoding means that the average voltage of the signal referred to the tissue potential is zero. In the case of exposure of tissue to the connection wires, average current leakage through tissue would also be zero.

While any suitable two-wire arrangement may be used, an example of one suitable two-wire connection containing two leads 52 and 54 and a connector 53 as depicted in FIG. 2 that may be used is as follows. The two-wire leads 52, 54 may consist of two insulated electrical conductive wires, with a DC insulation between each wire inside the main lead and the surrounding tissue is greater than about 1 Mohm, measured at VDC>100V. Each of the two wires may have a resistance of less than about 3 Ohm, including connector resistance. The capacitive load contribution is less than about 30 pF measured between two unconnected wires, including the connector capacitance. The total two-wire lead inductance is less than about 400 nH, or less than about 200 nH per wire. In one specific embodiment, the two wires may be made from 7 twisted strands of 0.152 mm diameter, 90% Platinum and 10% iridium. Each wire may be PTFE/FEP coated, and may be helically wound and inserted in a silicone tubing with a backfill of MED-6125 silicone. In one example, the DC insulation of the connector 53 between each contact point and the surrounding tissue is greater than about 50 Kohm

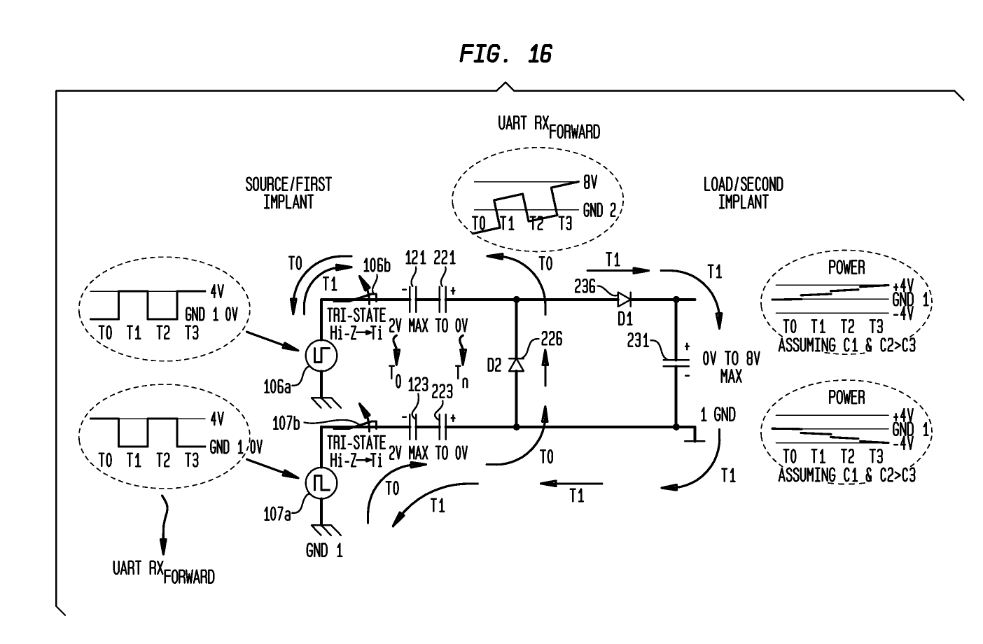

FIG. 16 shows a representation of the forward link in power and data transfer in the arrangement of FIG. 15. The tri-state switches 106b and 107b are closed during the transfer of power and data emanating from the first implant towards the second implant (forward link). The combined power and data signals generated by the logical gate inverters 106 and 107 are represented by two voltage sources 106a and 107a. Both sources generate a rectangular shaped signal which outputs are inverted to each other (balanced). In a first step at time T0 capacitors C121, C123, C221 and C223 are building up charge through conducting diode 226. In a second step (T1) the output voltage of the sources are inverted and diode 226 is non-conducting. At this time diode 236 is conducting and capacitor 231 is charged. The stimulator circuit 250 of the second medical implant 200, is seen as a load to the first medical implant 100. The load is connected in parallel to capacitor 231 which is a large smoothing capacitor also acting as a reservoir or a power storage device. The signal UART RX_{forward} on the cathode of diode 226 is clamped to gnd-1.

After an initial period (e.g. a few milliseconds), the voltage over the load is stabilized and the forward data (e.g. in a 8N1 UART format) can be received on the cathode of first diode 226 (UART RX_{forward}) without distortion or bit errors.

FIG. 16 also shows how the magnitude of stored power that is transferred across power storage device 231 increases to about 8V, having been doubled by voltage doubler 230 as previously described, as the transmission signal cycles through stages T0, T1, T2, T3 etc.

In another embodiment, medical implant system 500 may be provided with a backward data link functionality, to transfer data from the second medical implant 200 (or one or more other medical implants that may also be connected) to first medical implant 100. Such functionality may be used in a cochlear implant system when for example, the integrity of a cochlear implant is tested by sending test data to the implant, and receiving return data, representative of the integrity of the implant. Many other applications may also use the reverse link functionality.

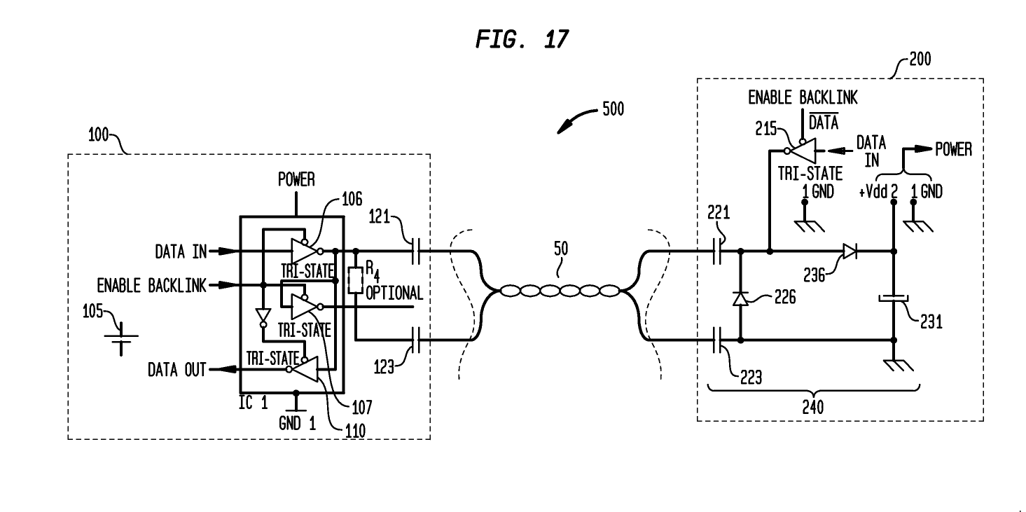

FIG. 17 shows medical implant system 500 comprising first medical implant 100 connected to a second medical implant 200 via two-wire connection 50. In one arrangement for providing back link functionality, data can be inserted to the voltage doubler through the cathode of diode 226 by use of a switch or tri-state buffer/inverter 215. In this example, to provide the back-link, the combined power and data transfer is interrupted (half-duplex) by changing the state of the tri-state buffer/inverters 106 and 110 in first medical implant 100 to high impedance. This allows retrieval of the data signal of the backlink by way of a tri-state buffer/inverter 110 in first medical implant 100.

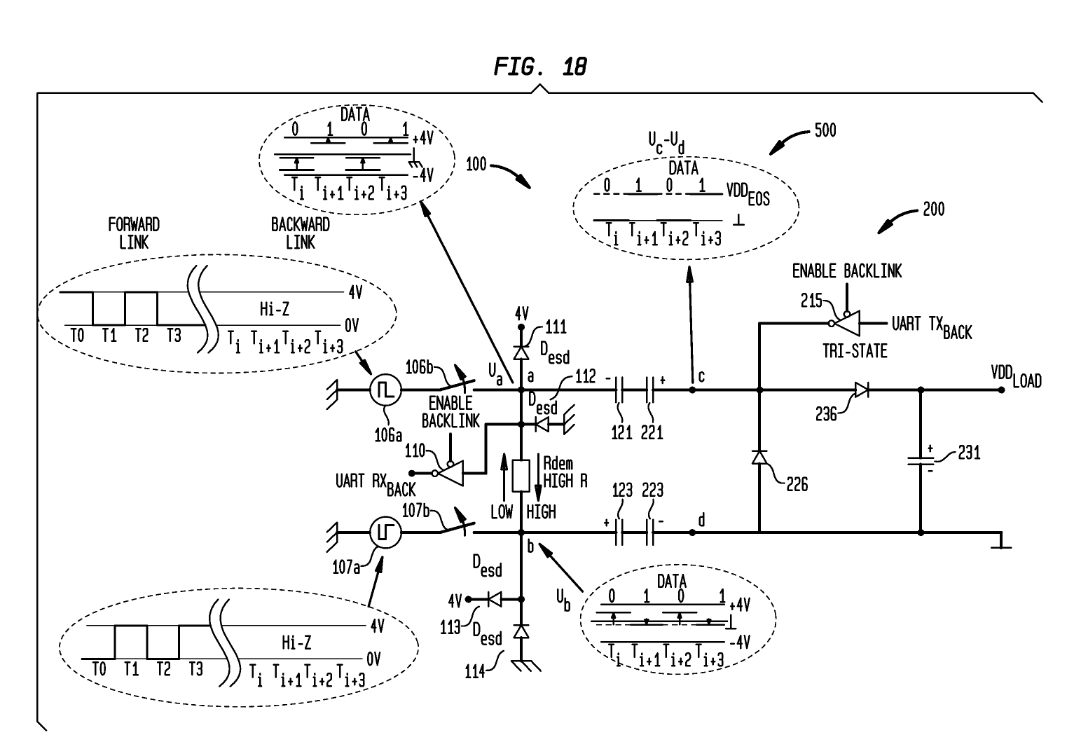

FIG. 18 shows the back link in the arrangement of FIG. 17, which in this example, occurs after the completion of the forward link described previously with reference to FIG. 16. The forward link tri-state drivers/inverters 106 and 107 are shown as source 106a and 107a in series with respective tri-state switches 106b and 107b of the first medical implant 100. During activation of the backlink the tri-state switches 106b and 107b are placed in a Hi-Z state or opened. The additional tri-state driver 215 of second medical implant 200 forces back link data (e.g. a logical 1) (VDD_{2nd}) or logical 0 (gnd) (see FIG. 18) over Rdem which is a high value resistor towards infinity. This data entry point on the second medical implant 200 is indicated as UART TX_{back} in FIG. 18. The received back link data is available on UART RX_{back}.

The tri-state driver output voltage is reflected over Rdem via DC decoupling capacitors 121 and 123. Electrostatic Discharge (ESD) diodes 111, 112, 113 and 114 in first medical implant 100 provide a clamping function. In this example, the back link is activated from the load/second medical implant 200 side but is initiated from the first medical implant 100/master side. It is assumed that the second medical implant is powered by the charge on capacitor 231 (power storage device) during the backlink.

Other methods of realising the backlink may include load modulation.

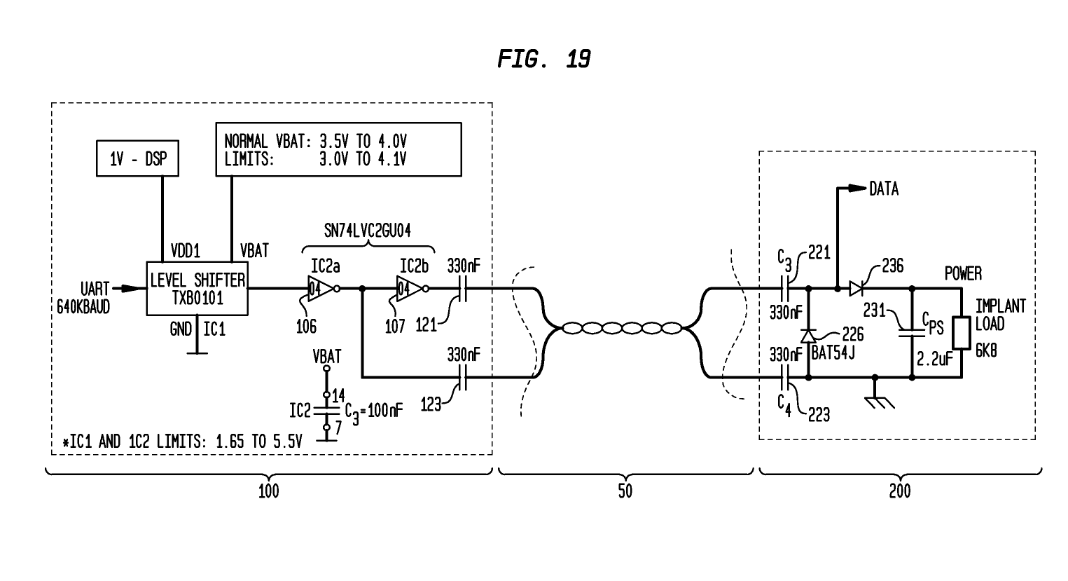

FIG. 19 shows a more detailed view of the arrangement of FIG. 15 (unidirectional power and data link), showing example component types and values. In particular, FIG. 19 shows that, in an embodiment, the inverter 106 and 107 is provided by an SN74LVC2GU04—TI Nanostar/Nanofree chip, DC decoupling capacitors 121, 123, 221 and 223 are X5R/X7R ceramic capacitors, 25V, of 330 nF+/−5%, and the first and second diodes, 226 and 236 are Schottky barrier diodes, type BAT54J.

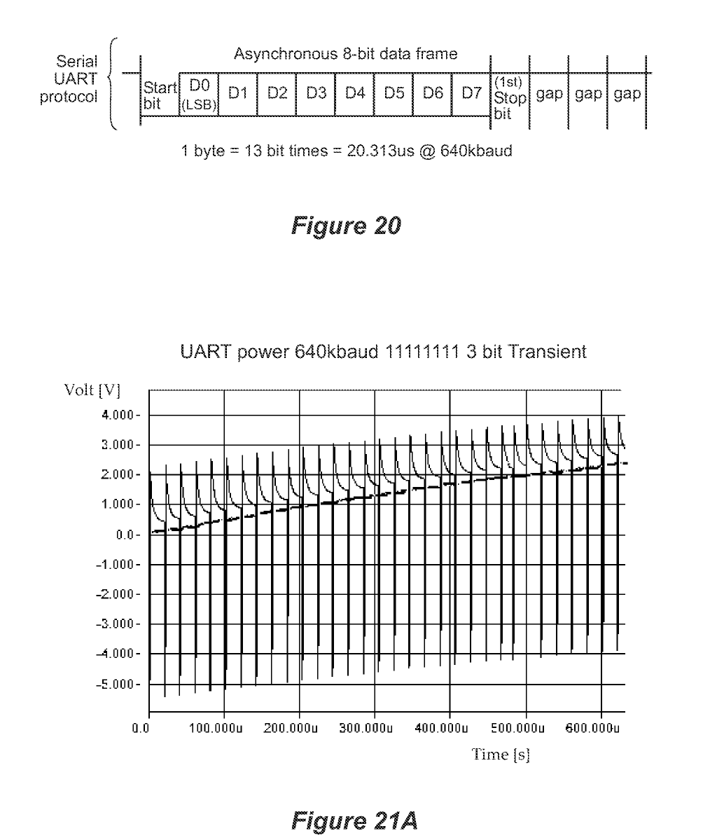

FIG. 20 shows an example of a suitable protocol for the forward link transmission. A 8N1 UART (Universal Asynchronous Receiver/Transmitter) data structure consists of a start bit, and then 8 data bits, terminated by a stop bit. After a predetermined gap, the next UART frame starts.

An example UART frame used to illustrate the operation of the arrangement of FIG. 19 is

Startbit (0)+11111111+stopbit (1).

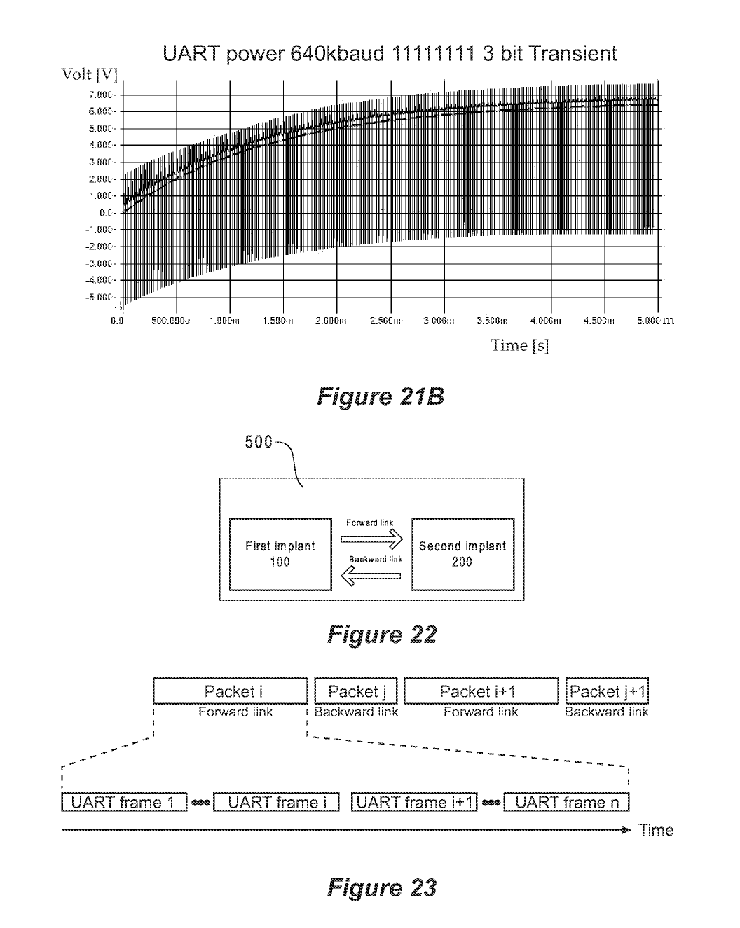

FIG. 21A shows the received data and power signal waveforms on the second medical implant 200 at start-up. The UART signal that is provided to the two-wire lead from the first medical implant 100 uses the data of FIG. 20 at a serial data speed of 640 kbps. The main waveform shows the data component, while the dotted line superimposed on this main waveform shows the signal over the power storage device component 231. This waveform is shown from the initial startup phase and so appears as inclining, as represented in FIG. 16.

FIG. 21B shows the same received and reconstructed signal in the second medical implant 200, seen on a larger timescale. Again, the main waveform is the data component, which is extracted for use by the second medical implant 200, and the dotted line superimposed is the signal over the power storage device 231, which is applied to the load or stimulator circuit 250. It can be seen that the voltage over the power storage device 231 becomes constant after about 4ms and the data can be restored on the cathode of the clamping or first diode 226 (see FIG. 19).

Forward link and backward data link between two implants 100, 200 in a medical implant system 500 as depicted in FIG. 22 can occur in an interleaved way on a two-wire link.

As shown in FIG. 23 multiple UART frames (10 bits+3 gap bits) can be transferred within a single forward packet i from the first implant to the second implant. Before a next forward data packet i+1 is transferred a backlink data packet may be transferred upon backlink activation as described previously.

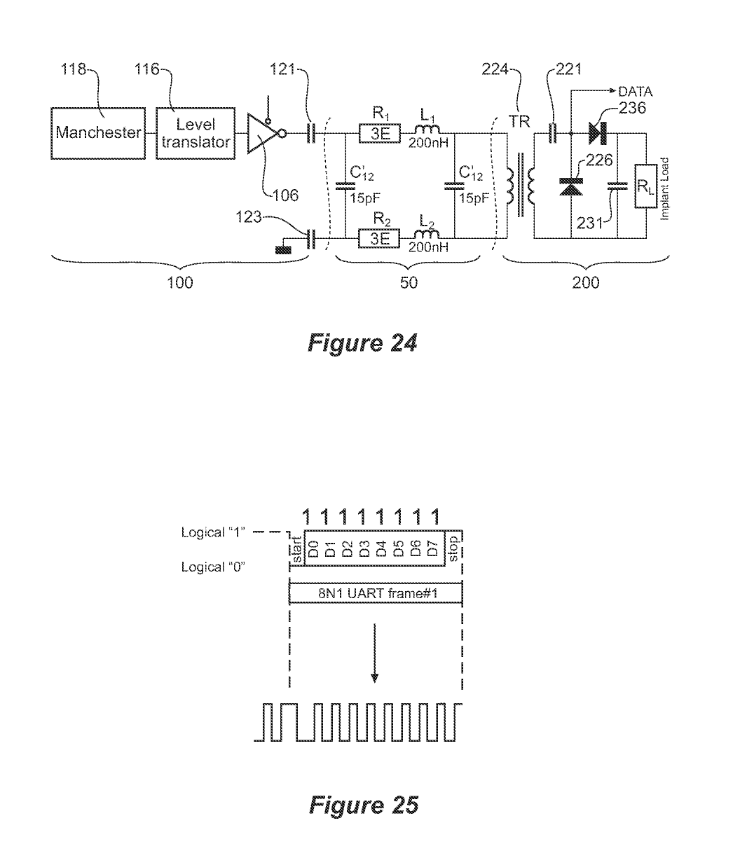

FIG. 24 shows a further alternative embodiment, expanding on the embodiment discussed previously with reference to FIG. 12, in which a transformer 224 is provided with second medical implant 200 to provide a wireless interface with two-wire connection 50. This may provide a DC decoupler in the form of a transformer. In this embodiment as shown in FIG. 24, second medical implant 200 comprises the clamping circuit provided by DC decoupling capacitor 221 and first diode 226, and voltage multiplier provided by the clamping circuit and second diode 236 and a capacitor 231. Data and power received on the signal via two-wire connection 50 may be extracted at the points shown in FIG. 24.

The advantage of clamping and extraction of power and data by the voltage doubler in the second implant is similar to that in FIG. 15. The transformer will change the incoming two-wire voltage and current if the turns ratio differs from 1.

In this figure, two-wire connection 50 is represented by the equivalent circuit with capacitance C′₁₂ (each of 15 pF), inductance L₁ and L₂ (each of 200 nH) and resistance R₁ and R₂ (each of 3 Ohm).

The first medical implant 100 comprises DC decoupling capacitors 121 and 123, driver 106, level translator 116 and Manchester coder 117.

As will be appreciated by the person skilled in the art, Manchester coding is a form of data communications line coding in which each bit of data is signified by at least one voltage level transition. This transition is low to high (0) or high to low (1). Time is divided into periods, and one bit is transmitted per period and the transitions signifying 0 or 1 occur at the midpoint of a period. Any transitions at the beginning of a period are overhead and do not signify data. These transitions that do not occur mid-bit do not carry useful information, and exist only to place the signal in a state where the necessary mid-bit transition can take place. The first half of a bit period is the true bit value and the second half is the complement of the true value.

Other forms of DC-free line coding that may be used in this embodiment include Bi-Phase Mark Line Code (BMC), Manchester Differential, Bipolar (polar RZ)—AMI, Bipolar—B8ZS, Bipolar—HDB3, 3B/4B block code, 8B/10B block code, and other scramblers such as Fibonacci and Galois scramblers. Each of these coding forms is know to the person skilled in the art.

In one form, this embodiment uses Manchester coding over the UART format. FIG. 25 shows an example Manchester IEEE 802.3 encoded UART frame for the data

Startbit (0)+11111111+stopbit (1)

Manchester frames offer very good power transfer efficiency for small sized cores and facilitates data recovery on the second implant, since saturation of the smaller core is less likely to occur due to its DC-free line coding and sufficient consecutive transitions for all data series.

It will be appreciated however, that the signal on the two-wire interface passing through a transformer does not need to be generated following a standard UART protocol including the start and stop bits. Any serial output on the first implant could generate Manchester encoded data without start and stop bits.

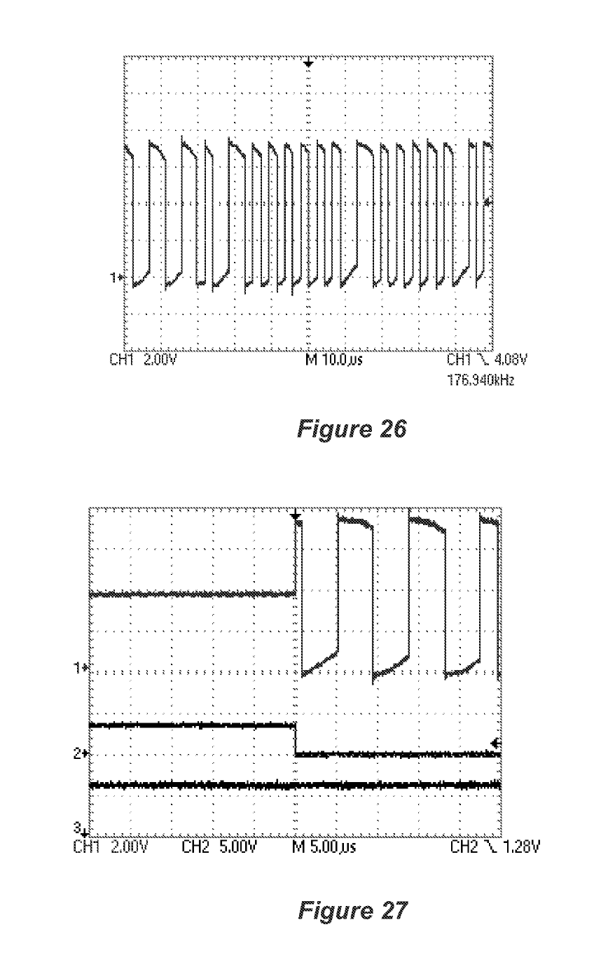

FIG. 26 shows the received waveform of the data signal on the second implant of the Manchester coded frame generated by first medical implant 100 for application to the two-wire connection 50 of FIG. 24. The data was derived from a Manchester encoded audio signal captured by microphone 102 (see FIG. 2 for example).

FIG. 27 shows the received waveform of the data signal at start-up on the second implant 200 for the start of the Manchester coded frame generated by first medical implant 100 for application to the two-wire connection 50 of FIG. 24. The data was derived from a Manchester encoded audio signal captured by microphone 102 (see FIG. 2).

A suitable transformer 224 as shown in FIG. 24 may be constructed using a torroidal core shape such as model numbers 11-540 and 11-580 provided by Ferronics Inc. The copper wire for the coils may be of two types. For the first type, the outer diameter of the wire may be about 0.04 mm and the outer diameter of the second type may be about 0.14 mm. Other transformer shapes are also possible such as the LPD4000 series provided by Coilcraft.

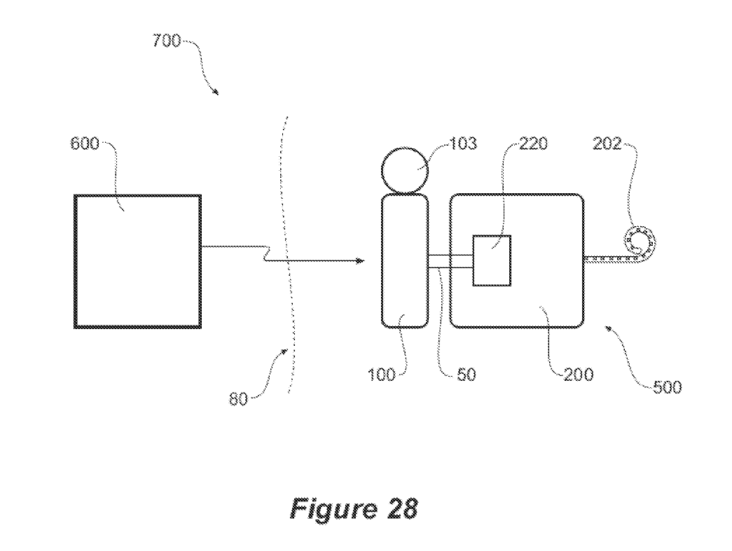

FIG. 28 shows a cochlear implant system 700 to which one or more of the various aspects described above may be used. The cochlear implant system comprises an external component 600, such as a processor. In use, processor 600 receives audio signals and converts the received audio signals into control signals as will be understood by the person skilled in the art. The processor 600 may also receive other types of signals such as test signals which are not necessarily provided as audio signals. Once converted, the control signals are then transmitted by the processor, for example, via an RF wireless transmitter coil (not shown).

The cochlear implant system 700 also comprises an internal component 500 for implantation in a user and for receiving the transmitted control signals. FIG. 28 shows internal component 500 implanted into the user, behind tissue 80.

In use, the generated control signals are transmitted through tissue 80 and are received by a receiving coil 103 of the internal component 500. As will be understood by the person skilled in the art, internal component 500 may be provided as a stimulator which in use, generates stimulation signals in accordance with the received control signals.

As shown in FIG. 28 the internal component comprises a first medical implant 100 which comprises a power source and the receiver 103 for receiving the control signals. The power source may be an onboard power source such as a battery 105, as shown in FIG. 2 for example, or the power source may simply be derived externally through the transmitted signal from the external component and extracted as a power component.

The internal component 500 also comprises a second medical implant 200.

A two-wire connection 50 connects the first medical implant 100 with the second medical implant. The two-wire connection is for transmitting a signal comprising a power component and a data component corresponding to the control signals between the first medical implant 100 and the second medical implant 200.

In this aspect, the second medical implant 200 comprises a clamping circuit 220 for extracting data received by the second medical implant 200 via the signal on the two-wire connection 50 and for rectifying the signal as described above. The second medical implant also generates stimulation signals in accordance with the control signals. In this aspect, the second medical implant also comprises a stimulator 202 for stimulating the user in accordance with the stimulation signals. In this example, stimulator 202 is a stimulating electrode for generating and applying the stimulation signals to the cochlea of the use to generate sound perception, simulating the audio signals received by the processor 600 as described above. In another example, stimulator 202 may be an actuator 300 of a DACS system as described above with reference to FIG. 2.

In one form, the second medical implant 200 may also have a power storage device 231 for storing power from the rectified signal, as shown in for example, FIG. 7 or FIG. 10.

In one form, the clamping circuit 220 forms part of a voltage multiplier circuit 230 for multiplying the signal received on the two-wire connection. In another form, there may also be provided a DC decoupler such as a DC decoupling capacitor which in one aspect, also forms part of the clamping circuit 220 as previously described.

Each of the aspects of the medical implant 200 and the medical implant system 500 may be applied to the cochlear implant system 700 of FIG. 28, in any combination as described above with reference to any one or more of FIGS. 1 to 28.

While the above has been described with reference to cochlear and hearing implants, it will be appreciated that the various aspects and variations may be applied to any suitable medical implant including cardiac stimulation implants, hormone regulation implants and other neural or muscular stimulation devices, including the following:

Auditory Brainstem Implant (ABI). The auditory brainstem implant consists of a small electrode that is applied to the brainstem where it stimulates acoustic nerves by means of electrical signals. The stimulating electrical signals are provided by a signal processor processing input sounds from a microphone located externally to the user. This allows the user to hear a certain degree of sound.

Functional Electrical Stimulation (FES). FES is a technique that uses electrical currents to activate muscles and/or nerves, restoring function in people with paralysis-related disabilities. Injuries to the spinal cord interfere with electrical signals between the brain and the muscles, which can result in paralysis.

Spinal Cord Stimulator (SCS). This system delivers pulses of electrical energy via an electrode in the spinal area and may be used for pain management.

Many variations and modifications may also be made within the scope of the present disclosure as will be understood by the person skilled in the art.

The reference to any prior art in this specification is not, and should not be taken as, an acknowledgement of any form of suggestion that such prior art forms part of the common general knowledge.

Throughout the specification and the claims that follow, unless the context requires otherwise, the words “comprise” and “include” and variations such as “comprising” and “including” will be understood to imply the inclusion of a stated integer or group of integers, but not the exclusion of any other integer or group of integers.

1. A medical implant comprising:
a clamping circuit for extracting data received by the medical implant via a signal on a two-wire connection and for rectifying the received signal to provide a rectified signal.

2. A medical implant as claimed in claim 1 further comprising a power storage device for storing power from the rectified signal.

3. A medical implant as claimed in claim 1 wherein the clamping circuit forms part of a voltage multiplier circuit for multiplying the signal received on the two-wire connection.

4. A medical implant as claimed in claim 1 further comprising a DC decoupler and wherein the DC decoupler forms part of the clamping circuit.

5. A medical implant as claimed in claim 4 wherein the DC decoupler comprises at least one DC decoupling capacitor.

6. A medical implant as claimed in claim 5 wherein the clamping circuit comprises the at least one DC decoupling capacitor connected to a diode.

7. A medical implant as claimed in claim 3 further comprising at least one DC decoupling capacitor which forms part of the voltage multiplier.

8. A medical implant as claimed in claim 7 wherein the voltage multiplier comprises the at least one DC decoupling capacitor connected to a first diode and a second diode.

9. A medical implant as claimed in claim 8 wherein the voltage multiplier is a voltage doubler.

10. A medical implant as claimed in claim 1 wherein the signal uses Universal Asynchronous Receive Transmit (UART) protocol.

11. A medical implant as claimed in claim 10 wherein the signal is generated without line coding.

12. A medical implant as claimed in claim 1 further comprising a transformer interface to the two-wire connection.

13. A medical implant as claimed in claim 12 wherein the signal is encoded using Manchester coding.

14. A medical implant as claimed in claim 13 wherein the signal uses Universal Asynchronous Receive Transmit (UART) protocol.

15. A medical implant system comprising:
a first medical implant comprising:
a power source; and
a data source,
a two-wire connection between the first medical implant and a second medical implant for transmitting a signal comprising a power component and a data component between the first medical implant and the second medical implant; and
the second medical implant comprising; and
a clamping circuit for extracting data received by the second medical implant via the signal on the two-wire connection and for rectifying the signal to provide a rectified signal.

16. A medical implant system as claimed in claim 15 further comprising a power storage device for storing power from the rectified signal.

17. A medical implant system as claimed in claim 15 wherein the clamping circuit forms part of a voltage multiplier circuit for multiplying the signal received on the two-wire connection.

18. A medical implant system as claimed in claim 15 further comprising a DC decoupler and wherein the DC decoupler forms part of the clamping circuit.

19. A medical implant system as claimed in claim 18 wherein the DC decoupler comprises at least one DC decoupling capacitor.

20. A medical implant system as claimed in claim 19 wherein the clamping circuit comprises the at least one DC decoupling capacitor connected to a diode.

21. A medical implant system as claimed in claim 17 further comprising at least one DC decoupling capacitor which forms part of the voltage multiplier.

22. A medical implant system as claimed in claim 21 wherein the voltage multiplier comprises the at least one DC decoupling capacitor connected to a first diode and a second diode.

23. A medical implant system as claimed in claim 22 wherein the voltage multiplier is a voltage doubler.

24. A medical implant system as claimed in claim 15 wherein the signal uses Universal Asynchronous Receive Transmit (UART) protocol.

25. A medical implant system as claimed in claim 24 wherein the signal is generated without line coding.

26. A medical implant system as claimed in claim 15 further comprising a transformer interface to the two-wire connection.

27. A medical implant system as claimed in claim 26 wherein the signal is encoded using Manchester coding.

28. A medical implant system as claimed in claim 27 wherein the signal uses Universal Asynchronous Receive Transmit (UART) protocol.

29. A cochlear implant system comprising:
an external component for receiving audio signals and for converting the received audio signals into control signals and for transmitting the control signals; and
an internal component for implantation in a user and for receiving the transmitted control signals and for generating stimulation signals in accordance with the received control signals, the internal component comprising:
a first medical implant comprising:
a power source; and
a receiver for receiving the control signals,
a two-wire connection between the first medical implant and a second medical implant for transmitting a signal comprising a power component and a data component corresponding to the control signals between the first medical implant and the second medical implant; and
the second medical implant comprising:
a clamping circuit for extracting data received by the second medical implant via the signal on the two-wire connection and for rectifying the signal to provide a rectified signal; and
a stimulator for stimulating the user in accordance with the stimulation signals.

30. A cochlear implant system as claimed in claim 29 wherein the second medical implant further comprises a power storage device for storing power from the rectified signal.

31. A cochlear implant system as claimed in claim 30 wherein the clamping circuit forms part of a voltage multiplier circuit for multiplying the signal received on the two-wire connection.

32. A cochlear implant system as claimed in claim 31 further comprising a DC decoupler and wherein the DC decoupler forms part of the clamping circuit.

33. A medical implant comprising:
a power storage device for storing power received by the medical implant via a signal on a two-wire connection; and
a clamping circuit for extracting data received by the medical implant via the signal on the two-wire connection.

34. A method of processing a signal comprising a data component and a power component on a two-wire connection of a medical implant, the method comprising:
receiving the signal on the two-wire connection;
rectifying the signal using a rectifier to extract the power component; and
clamping the signal using the rectifier to extract the data component.
